(19) United States
(12) Patent Application Publication
(10) Pub. No.: US 2022/0008764 A1
(43) Pub. Date: Jan. 13, 2022

(54) All season Non CO2 magnetic filtered multilayered face mask for humans and animals

(71) Applicant: Keith Ray Elam, Eugene, OR (US)

(72) Inventor: Keith Ray Elam, Eugene, OR (US)

(21) Appl. No.: 16/926,706

(22) Filed: Jul. 12, 2020

(51) Int. Cl. A62B 23/02; A41D 13/11; A41D 1/00; A41D 31/10; A41D 31/08; A41D 31/06; A41D 5/00; A41D 31/30; B32B 5/02; B32B 5/26; B32B 7/09; A41D 27/20; A62B 18/10; A62B 18/06; A62B 18/00; A61F 11/14; A62B 18/08

(52) U.S. Cl. CPC A62B 23/025; A41D 2300/32; A41D 1/002; A41D 31/10; A41D 31/085; A41D 31/065; A41D 5/00; A41D 13/1192; A41D 31/305; B32B 5/024; B32B 5/26; B32B 7/09; A41D 27/20; A62B 18/10; A62B 18/06; A62B 18/003; A61F 11/14; A62B 18/084; B32B 2571/00; B32B 2262/065; B32B 2262/08; B32B 2250/04; A41D 2400/48; A41D 13/1161

(57) ABSTRACT

A multilayered hemp face mask for filtering air particulate matter which has velcro loop and hook fabrics integrated with each of the fabric layers of the mask, the mask comprising: at least one DC fan, at least one copper rivet and a magnet, at least two fabric one way valves and a pair of ear muffins.

### CROSS REFERENCE TO RELATED INFORMATION

This application is a continuation of U.S. patent application Ser. No. 16/893,275, filed on Jun. 4, 2020, the contents of which are hereby incorporated herein in its entirety.

### FIELD OF THE INVENTION

The present invention relates to universal face masks and more particularly to face masks made from hemp fibre. It further relates to face masks having multiple layers of fibres for filtration of the air pollutants and which can be made for use by the humans as well as designed to be worn by animals especially dogs and horses.

### BACKGROUND OF THE INVENTION

Face mask has long been considered indispensable in the spread of infection by preventing airborne infections of the wearer by absorbing droplets, when someone coughs, talks or sneezes, close-by. Thus, face masks with ability to eliminate cross-contamination, can be a pivotal element in infection control strategy of a society.

In recent years, the emergence of deadly viruses with contagion properties are spelling havoc in our societies, resulting in severe straining of the healthcare systems as also of the economies. Surging morbidity rates by potentially dangerous viruses as likes of SARS (severe acute respiratory syndrome) and avian influenza, Ebola, H1N1 and the recent COVID 19 are dooming our future and putting our civilization to the brink of destruction. The earlier developed masks, suffered from the problem of secondary infection on account of viruses adhering to the used mask and contaminating hands during disposal/changing. Therefore, a mask is of little use in preventing infection. Efforts to take care of the deficiency led to new developments thereby inspiring inventors/community health specialist to relook at developing masks that can control the spread of viruses in highly effective and eco-friendly manner. A number of masks have been developed in line with complete avoidance of secondary infections.

### PRIOR ART

CN101946990A titled, “Nano silver selenium negative ion anti-bacterial face mask for controlling H1N1 influenza” relates to a nano silver selenium negative ion anti-bacterial face mask for controlling H1N1 influenza, belonging to the technical field of the production of health textile engineering fabrics face masks. At present, the H1N1 influenza globally grinds on, dire threats are brought for the life and the health of human beings, once the H1N1 influenza largely grinds on within the globe, a plenty of human lives can be captured possibly, and the death toll even exceeds any one World War. The H1N1 influenza is caused by an influenza virus; test researches shows that no microorganism can not be killed by silver within 6 min, thus the silver is a known antibacterial agent capable of killing all bacteria and viruses. Selenium, germanium and honey serial products capable of enhancing the human immunity, and nano mixed powder of medical stone, tourmaline and traditional Chinese medicine formations are added into fiber fabrics of a spinning solution to prepare the nano silver selenium negative ion anti-bacterial face mask for controlling H1N1 influenza which can achieve the purpose of no treatment of diseased patients but preventive treatment of diseases, wherein the nano mixed powder is capable of releasing trace elements and negative ions.

US20160113336A1 titled, “Face mask” discusses a mask that has a mask body and a pair of ear straps. The mask body includes an outer layer sheet and an intermediate layer sheet. The outer layer sheet is formed of hydrophobic fibers. The intermediate layer sheet is laid on the outer layer sheet so as to be located on a wearer's side of the outer layer sheet when the mask is worn. The intermediate layer sheet includes a first fiber layer which is formed of polyolefin fibers containing an inorganic antimicrobial agent and a second fiber layer which is formed of polyolefin fibers and has a larger fiber diameter than the first fiber layer. The fiber diameter of the first fiber layer is within a range of 0.5 to 2.8 μm and the ratio of a particle diameter of the inorganic antimicrobial agent with respect to the fiber diameter is within the range of 0.1 to 6.0.

WO2009026725A1 titled, “Antimicrobial compositions and fibres incorporating the same” defines an antimicrobial composition comprising (a) at least two antimicrobial agents having different antimicrobial mechanisms of action and being present in amounts that together provide a synergistic antimicrobial effect or (b) an antimicrobial agent and a surface modifying agent, an antimicrobial masterbatch comprising antimicrobial composition (a) or (b) and a polymer carrier, an antimicrobial fibre composition comprising the antimicrobial masterbatch and a fibre substrate, an antimicrobial fibre comprising a fibre body or a fibre surface having the antimicrobial fibre composition, and a process for producing antimicrobial fibres.

EP2969040A1 titled, “A facemask having one or more nano fiber layers” talk of facemasks and methods utilizing nano fibers. The facemask and methods provided reduce pressure drop and enhance filtration of airborne particles. In one embodiment there is a facemask, comprising: an inner layer comprising nonwoven fiber material configured to contact a wearer's nose and mouth; a middle layer disposed on the inner layer and comprising a nanofiber material; an outer layer disposed on the middle layer and comprising nonwoven fiber material, the facemask having upper and lower edges and at least one pleat configured to fit over a chin of the wearer when unfolded, the at least one pleat configured for folding and unfolding the facemask such that when folded the facemask is configured to assume a generally flat storage configuration and when unfolded the facemask is configured to cover at least the nose and mouth of the wearer.

Whereas, in all the prior arts specified, novel methods to take care of secondary infection using advanced fabrics and chemicals in masks are specified, but usage of artificial fabrics/chemicals entails limit to their usage. In such a scenario, use of a material that is natural as well as having appropriate properties to prevent secondary infection is considered more trustworthy. The present invention is an effort, in that direction.

### SUMMARY OF THE INVENTION

An aspect of the present invention is to provide a face mask for humans as well as for animals that is primarily made of hemp fibre.

A further aspect of the invention is to provide a face mask with at least 2 or more consecutive layers.

A still further aspect of the invention is to provide a mask which is reusable and washable without damage or change in shape.

Another aspect of the invention is to provide a face mask which is biodegradable.

A still further aspect of the present invention is to provide a face mask which fits not only snugly but also comfortably against the side of the face.

Another aspect of the invention is to provide a facemask which allows breathing without restriction.

A still another aspect of the invention is to provide a face mask which is adapted to fit over the nose, mouth and chin of the wearer and to conform at its edges reasonably closely to the face and neck of the wearer.

In one aspect, the invention is directed to a face mask comprising an outer layer, a second layer, a third layer and an inner layer.

In an aspect of the invention, the outer most layer filters out the heavy particles in the atmosphere. This layer is made of hemp fibre that is woven into a semi-coarse hemp fabric.

In another aspect of the invention, the outer layer of the said face mask may be woven into many different types of patterns, like square or pinwheel designs and may also include one or more images of flowers, animals and the like, any custom designs like animal faces, fish faces or cartoon characters giving the face mask a fashionable twist, thereby encouraging the mass to wear face masks voluntarily for their protection. Government Emblems can also be embroidered to the face mask to allow the Mass to be able to recognize Government officials during Emergency Crisis while they are having to wear face masks.

In still another aspect of the invention, the second layer may be of thick hemp fibre fabric which may be used for absorbing water for better filtration or for keeping the face warm in cold weather when the hemp face mask is dry.

In another aspect of the invention, the third layer of the hemp face mask may be of dense and a durable fabric hemp fabric which is, made from a double strand of long-fibre hemp which is wet-spun yarn, woven in a plurality of directions, causing the hemp canvas fabric to become the densest.

In still another aspect of the invention, the fourth layer which is the innermost layer and touches the skin of the wearer may be made either from 100% hemp fibre, or hemp and bamboo fibre or hemp and silk fibre or 100% silk.

In further aspects of the invention, the hemp face mask can have more hemp fabric layers added on or fewer fabric layers by removing some layers, depending on the environment the hemp face mask is to be used for.

In a still further aspect of the invention, the four layers of fabric of the hemp face mask are attached with each other by means of sowing.

In further aspects of the invention, a pocket is sewn onto on to the inner layer of the hemp face mask which allows to store items while wearing the hemp face mask, or to tuck the other side of the hemp mask inside the pocket.

In some aspects of the invention, two pairs of drawstrings namely are provided with the body of the mask which are pulled up together and locked together using locking means, to fit the hemp face mask, on the head tightly.

In another aspect of the present invention, the drawstrings are braided from natural hemp fibre; wet-spun hemp yarns which could feature a hollow core. These drawstrings are soft and flexible and perfect for the use with the hemp face mask.

In some other aspects of the invention, the drawstrings may be a one piece hemp drawstring braided from natural hemp fibre with wet-spun hemp yarns which could feature a hollow core.

In a still further aspect of the invention, a hemp fabric padding may be provided for supporting the hemp drawstrings on the head and neck of the wearer while protecting the head, hair and neck of the wearer from rubbing of the drawstrings.

In still further aspects of the invention, the hemp face masks may be designed to be worn by the animals especially dogs and horses to provide more Oxygen to the animals, prevents insects from entering the nose of the animals and also helps in, filtration of particulate matter as well as from other dog bites apart from keeping the animal's face warm during winters or cool during the summers.

These and other embodiments of the subject invention will readily occur to those of skill in the art in light of the disclosure herein.

### BRIEF DESCRIPTION OF THE DRAWINGS

FIG. 1 is a rear view of an exemplary hemp face mask described herein.

FIG. 2A is a side view of the mask of FIG. 1.

FIG. 2B is an enlarged view of the four layers of hemp fabric utilized in the making of the mask.

FIG. 3A is a perspective view of another exemplary embodiment of the present invention depicting face masks for animals.

FIG. 3B is an exemplary embodiment of animal face mask depicted as worn by an animal with a fan, a power source, an ampere fuse and a dimmer switch.

FIG. 4A is an enlarged view depicting the positioning of the copper rivet along with the pouch for the magnet and the placing of the magnet in the inner layers of the face mask.

FIG. 4B is a Pocket used to store one side of the Face Mask, can also be used for a one way valve for wither inlet or outlet holes and depicts the positioning of the hook and loop Velcro to remove or attach to the outer layers of the face mask. FIG. 4B can also be used to store a small DC Battery when Face Mask is worn.

FIG. 4C Is the back side of FIG. 4A depicting a copper rivet, a sleeve, pouch and magnet to be used as an inlet or outlet valve.

FIG. 5 is an exemplary embodiment depicting a pair of fans, copper rivets and magnets in the face mask of the present invention.

FIG. 6 is an exemplary embodiment depicting the ear muffins of the present invention.

### REFERENCE NUMERALS

| | |
|---|---|
| 10 Left hand side of the face mask | 20 Left hand side of the face mask |
| 30 Fabric Stitch | 40 Double Stitch and a Double |
| | Fold |
| 50 Second Sew of the Hemp Fabric | 60 Pocket |
| 70 Pouch | 80 Spring |
| 90 Holes | 100 Hemp Fabric Padding for the |
| | head region |
| 110 Hemp Fabric Padding for the | |
| neck region | |
| 120, 130 parts of Hemp Drawstring | |
| to be wound around the head | |
| 140, 150 parts of hemp drawstring | |
| to be wound around the neck | |
| 125 One Piece Hemp Drawstring | |
| for the head | |
| 135 One Piece Hemp Drawstring | |
| for the neck | |
| 145 Stitching that Holds 125, 130, | |
| 135, 140 with the face mask | |
| 155 Stitching Cover | |
| 160 Bottom of a Toggle Lock | 170 part of toggle lock |
| 180 Design, Logo to be stitched | 190 First Layer of the Hemp |
| | Face Mask |
| 200 Second layer | 210 third layer |
| 220 Fourth layer | 230 Front face of the Hemp |
| | Animal Face Mask |
| 240 Hemp Drawstring Cord for | |
| winding over the Center of the | |
| Head of the animal face | |
| 250 Hemp Drawstring Cord that | |
| for tying onto the Bottom of the | |
| Collar of the animal face | |
| 125, 135 Drawstrings to be cut | |
| and Toggle Locks removed or | |
| additional added. | |
| 251 Velcro Loop Fabric | 252 Velcro Hook Fabric |
| 255 Copper Hole Rivet | 256 Sleeve/One way valve |
| 260 Magnet Pouch | 270 Magnet |
| 280 Velcro Fabric | 290 Velcro Fabric |
| 300 Cover with Velcro | 310 Velcro Fabric |
| 315 Velcro Fabric | 320 Pouch to hold DC Brushless |
| | Motor and Fan |
| 330 DC Brushless Motor and Fan | |
| 340 DC Positive and Negative Wires | |
| 350 DC Fuss | 360 DC Power Source |
| 365 Dimmer Switch | 370 DC Plugs |
| 380 Female Brass Button | 390 Male Brass Button |
| 400 Ear Muffins | 410 Outer Layer of the Ear |
| | Muffins |
| 415 Second Layer of the Ear Muffin | |
| 420 The inner layer of the | |
| Ear Muffins | |

### DETAILED DESCRIPTION OF THE EMBODIMENTS

Face masks as described herein find use in various indications, including in health care, surgery, dental applications, research labs, clean rooms, construction sites, air travel, veterinarian applications, cosmetology, environmental settings and the like. Methods of making and using these masks also form aspects of this invention.

The recent spread of COVID 19, has initiated the use of face masks for each and every person to prevent the spread of the virus. In such a scenario, the use of cloth face masks is an alternative, provided, according to FDA, the following points are taken into consideration.

The mask must cover the nose and below the chin;

It must fit not only snugly but also comfortably against the side of the face;

It must be secured with ties or ear loops;

It must include multiple layers of fabric;

It must allow for breathing without restriction; and

It must be able to be laundered and machine dried without damage or change in shape.

The above properties can be found with cotton fabric and cotton face masks and are available in the market easily. However, all these properties are more evident in face masks made from hemp fabric as well. Hemp fabric is a type of textile that is made using fibres from the stalks of the Cannabis sativa plant which has been recognized as a source of extraordinarily tensile and durable textile fibres.

The stalks of the hemp plant consist of two layers: The outer layer is formed from rope-like bast fibers, and the inner layer consists of a woody pith. For textile purposes only the outer layer of the Cannabis sativa stalk is used. Once the outer layer of bast fibers is stripped from the hemp plant, it can be processed and made into rope or yarn. Hemp rope is strong and remains renowned as an excellent material for clothing that surpasses cotton and synthetic textiles by most metrics.

Once hemp is processed into fabric, it has a similar texture to cotton, but it also feels somewhat like canvas. Hemp fabric is not susceptible to shrinkage, and it is highly resistant to pilling. Since fibres from this plant are long and sturdy, hence hemp fabric is not only very soft, but it is also highly durable.

Since hemp is a lightweight fabric, it is highly breathable, and it also effectively facilitates the passage of moisture from the skin to the atmosphere, and is, therefore ideal for hot climates. It is easy to dye this type of fabric, and it is highly resistant to mould, mildew, and potentially harmful microbes.

Hemp fabric softens with each washing, and its fibers don't degrade even after dozens of washings. Further, hemp can be blended with cotton as well as blended with silk. Blending hemp with other fabrics can make this textile softer while remaining durable.

The above qualities of hemp fibre, makes it ideal for making face masks.

The present invention, in view of the above properties of hemp fibre, utilizes it for making of masks.

The terms “hold,” “join,” “secure,” “attach” and derivatives and synonyms thereof refer to any affixing of such structure(s) of the present invention to another structure(s) and may be accomplished by any of several conventional methods. Thus, drawstrings may be secured by use of sewing, by tying or any other suitable mechanism that holds the mask in place. By way of example and not limitation, these methods include stitching, gluing, heat sealing etc.

First, the general configuration of a mask for humans of the first embodiment will be described. The mask of the first embodiment includes a mask body 1 having a substantially oval shape and symmetrically and longitudinally divided into two halves—the left and the right, the left side of the hemp mask is denoted by 10 and the right side by 20.

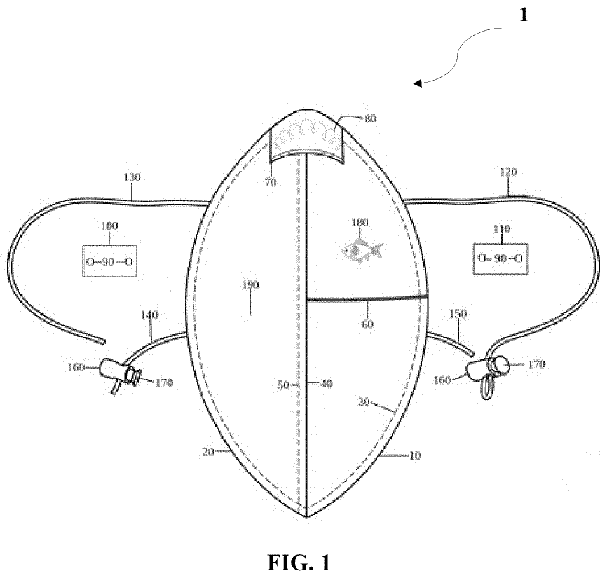

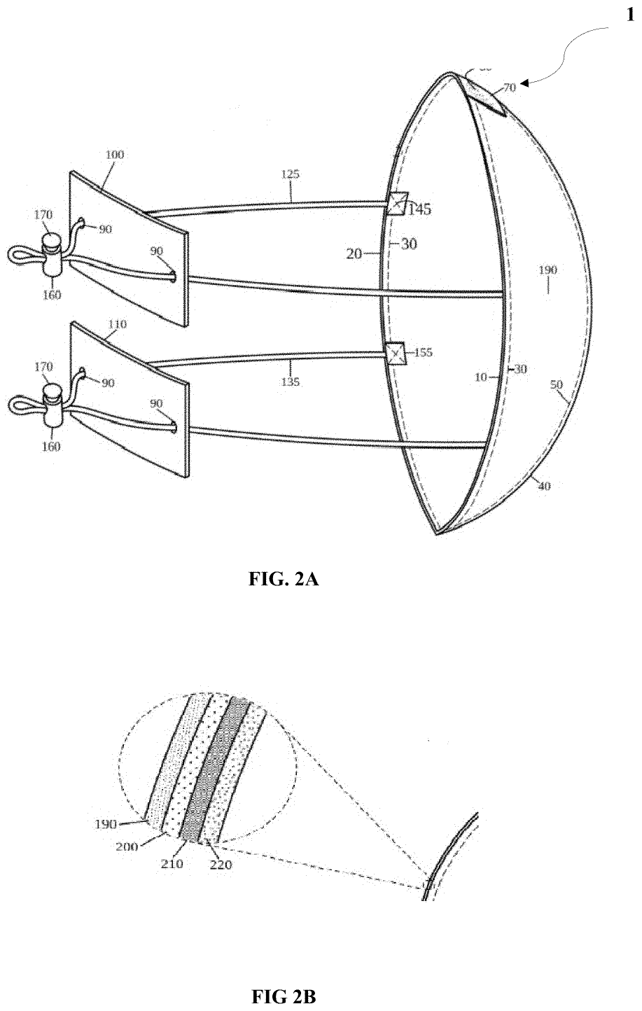

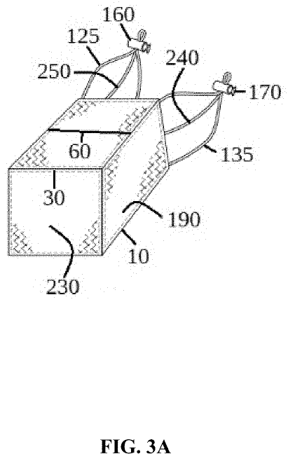

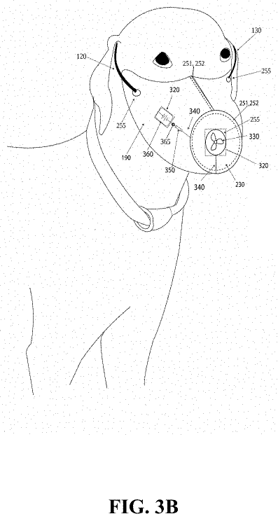

FIGS. 1 and 2 are the rear and side view depictions, respectively, of an exemplary hemp fabric face mask according to the present invention. The mask, is a substantially oval shaped woven hemp fabric material, comprising of four layers of hemp fabric, depicted in FIG. 3 as 190, 200, 210 and 220.

The first layer 190 or the outer most layer, visible to the public eye filters out the heavy particles in the atmosphere. This first layer is made of hemp fibre that is woven into a semi-coarse hemp fabric. In some embodiments of the invention, the first layer of the hemp mask 190 the hemp fibre may be woven into many different types of patterns, like square or pinwheel designs. In some other embodiments, the mask may also include one or more images of flowers, animals and the like, any custom designs like animal faces, fish faces or cartoon characters. This allows the face mask of the wearer to stand out in public giving the face mask a fashionable twist, thereby encouraging the mass to wear face masks voluntarily for their protection. Government Emblems can also be embroidered to the face mask to allow the Mass to be able to recognize Government officials during Emergency Crisis while they are having to wear face masks.

The second layer 200 is a thick hemp fibre fabric which may be used for absorbing water for better filtration or for keeping the face warm in cold weather when the hemp face mask is dry.

The third layer 210 of the hemp face mask comprises of hemp fabric which is dense and a durable fabric, made from a double strand of long-fibre hemp which is wet-spun yarn, woven in directions, causing the hemp canvas fabric to become the densest. Since, the plied hemp yarns are both warp and weft, the hemp fabric of the third layer is very thick. The third layer 210 of the hemp face mask filters out air particles which the outer layer 190 and second layer 200 are unable to filtrate. Since the hemp canvas fabric of the third layer 210 becomes denser when wet, it is more adept to filter out air particles like coal dust.

The fourth layer 220 is the innermost layer which touches the skin of the wearer. The innermost layer 220 can either be made from 100% hemp fibre, or hemp and bamboo fibre or hemp and silk fibre or 100% silk.

In some embodiments the hemp face mask can have more hemp fabric layers added on or fewer fabric layers by removing some layers, depending on the environment the hemp face mask is to be used for.

The four layers of fabric 190, 200, 210 and 220 are attached with each other by means of sowing. The various rounds of stitches are denoted by numerals 30, 40 and 50 in FIG. 1. 30 is the fabric stitch which holds the four layers of the hemp fabrics 190, 200, 210, 220 together. While stitching the ends of the hemp fabric will be rolled once, then rolled and again and sown together as one piece. As described above the main body of the hemp face mask is symmetrically divided into two parts left 10 and right 20. During stitching these two parts 10 and 20 respectively are also stitched as described and sown together as one piece. This double fold and stitch prevents the hemp fabric from shredding apart. 40 thereby denotes the double stitch and double fold of the sewing of left 10 and right 20, the two parts of the face mask. Then again stitch and fabric is rolled once, and then rolled twice. Then sown again denoted by 50 in FIG. 1. This method prevents the first sown threads that allow materials in the atmosphere to pass through the first sew of the hemp fabric.

A pocket 60 is sewn onto on to the inner 190 layer of the hemp face mask on the left side 10 of the mask. The pocket 60 allows to store items while wearing the hemp face mask, or to tuck the other side of the Hemp Mask inside the pocket 60.

A small pouch 70 is sown at the upper part of the oval face mask which coincides with the bridge of the nose, for holding a small spring 80. The small spring 80, is heated up in the center and bent into a V-Shape for using as a nose support and also helps in giving shape and form to the hemp face mask around the bridge of the nose, for better protection from the materials and particles in the atmosphere to enter into the hemp face mask.

In some embodiments, a logo, design, or name 180 can be sown into the hemp face mask pocket 60.

In an embodiment of the present invention, two pairs of drawstrings namely 120, 130 and 140, 150 (corresponding to members used when the mask is worn) are provided with the body of the mask. Referring to FIG. 1, drawstring 120 is fitted at the top left hand side of the hemp face mask and drawstring 130 is sown at the top right hand side of the hemp face mask. The drawstrings 120 and 130 are pulled up together and locked together using locking means or the use of a slipknot, to fit the hemp face mask, on the head tightly.

Similarly the drawstrings 140 and 150, are sown and fitted at the bottom right hand side of the hemp face mask and bottom left hand side of the hemp face mask respectively and 140 and 150 are pulled up together and locked together using locking means, to fit the hemp face mask or the use of a slipknot, to fit the hemp face mask, on the neck tightly.

These drawstrings 120, 125, 130, 135, 140 and 150 are braided from natural hemp fibre; wet-spun hemp yarns which could feature a hollow core. These drawstrings are soft and flexible and perfect for the use with the hemp face mask.

Generally, the locking means is a toggle lock comprising the bottom of the toggle lock 160 housing a spring for the knob 170 for pushing down to unlock the toggle for adjusting 130 and 140 in place, along with the other toggle lock for 140 and 150 to go through respectively. The locking means is used to adjust the hemp drawstring cord to allow them properly to be fitted on the neck and the top of the head respectively.

In another embodiment, a pair of drawstrings can be provided instead of two pairs. Referring to FIG. 2, a one piece hemp drawstring 125 braided from natural hemp fibre with wet-spun hemp yarns which could feature a hollow core. The draw-string is soft and flexible and perfect for the use for the hemp face mask and is positioned, near the upper part of the hemp face mask and locked together using locking means comprising the toggle lock 160 and 170, to fit the hemp face mask, on the head tightly.

Similarly, another one piece hemp drawstring 135 also braided from natural hemp fibre with wet-spun hemp yarns which could feature a hollow core. The draw-string is soft and flexible and perfect for the use for the hemp face mask and is positioned, near the lower part of the hemp face mask and locked together using locking means comprising the toggle lock 160 and 170, to fit the hemp face, on the neck tightly.

A hemp fabric padding 100 supports the hemp drawstrings 130 and 120 on the head of the wearer and also protects the head and hair from rubbing of 160 and 170 against the head of the wearer.

Similarly another hemp fabric padding 110 supports the hemp drawstrings 140 and 150 on the neck of the wearer and also protects the neck from rubbing of 160 and 170 against the neck of the wearer.

The hemp fabric padding 100 is provided with a pair of holes 90, 90′ for allowing the drawstrings 130 and 120 to pass through which gets adjusted with the fastening means to be snugly fitted over the head. Similarly the hemp fabric padding 110 is provided with a pair of holes 95, 95′ for allowing the drawstrings 140 and 150 to pass through which gets adjusted with the fastening means to be snugly fitted over the neck region of the wearer.

In an embodiment of the present invention the hemp fabric drawstrings is fastened with the body of the mask near the edge by sewing it with the layer of fibres. The sowing is done either with the top layer 190 or with the inner most layer of the hemp fabric 220 or in between two fabric layers 200 and 210.

In the event that the drawstrings are sown with the innermost layer of 190, a small fabric 155 with stitching 145 is sown over the stitched drawstrings 125, 135, 130, 120, 140 and 150 mainly for providing a smooth cover against the rubbing of the drawstrings with the facial skin of the wearer and additionally to provide aesthetic covering to the sown drawstrings.

In some embodiments of the present invention, the hemp face masks can be designed to be worn by the animals especially dogs and horses. The hemp mask for animals not only prevents insects from entering the nose of the animals but also helps in filtration of particulate matter as well as from other dog bites apart from keeping the animal's face warm during winters.

The animal mask of the embodiment is depicted in FIG. 3A which is cuboidal in shape and has five faces. The hemp drawstring cord 240 is positioned over the centre of the head for tying to the collar or the horse bridal for securing the hemp face mask. Whereas the hemp drawstring cord 250 is used for tying onto the bottom of the collar of horse bridal to secure the hemp face mask in place. 230 depict the frontal face of the hemp animal face mask. The parts of the toggle locks 160, 170 functions for tightening the cords of the animal face mask with the animal face. The cords 125, 135 provided, can be cut for either removing the toggle locks or for adding more toggle locks for adjusting and securing the animal face mask on the animal's face.

The hemp face mask for animals are made of the same layers of fibres and sown in the same as is described for the human face masks.

Since long term usage of masks may lead to accumulation of CO₂ within the mask cavity that may be harmful to the user causing headaches and dizziness, hence, to overcome this drawback as is evident in the face masks of prior arts, the hemp face mask has introduced certain modifications.

In some embodiments, the multilayered hemp face mask comprises of layers with Velcro loop and hook fabric which enables each of the layer to be interchanged or flipped and the number of layers reduced depending upon the requirement of the user and the applicable environment.

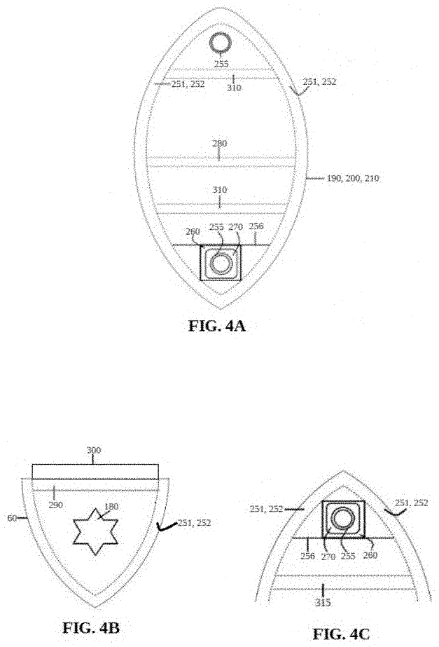

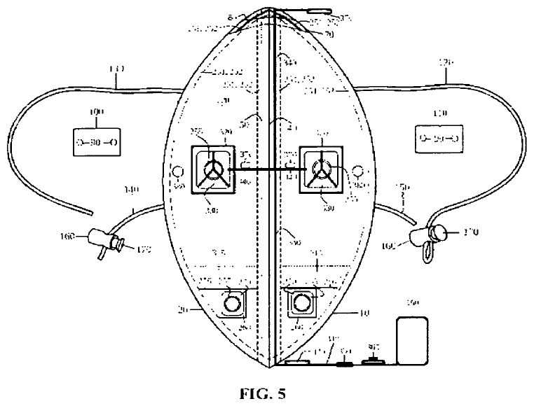

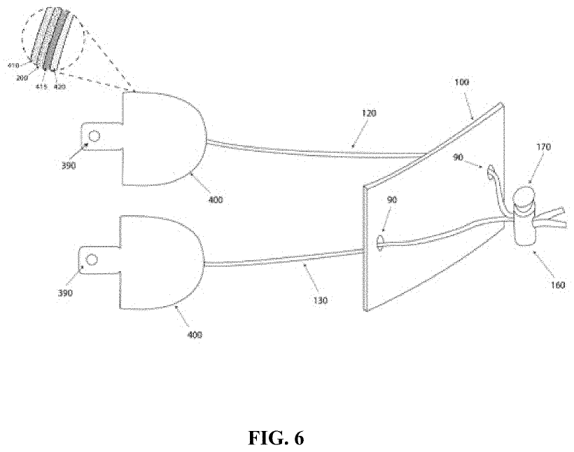

Referring to FIG. 4 and FIG. 5, the Velcro loop and hook fabric are denoted by 251 and 252 respectively which helps in flipping of the layers or in interchanging of the multiple layers onto the face mask.

In some embodiments of the present invention the face mask can be modified to incorporate a miniature DC fan to allow inhalation of more air and oxygen and also allowing quick removal of CO₂ from the mask through enhanced circulation.

To incorporate the above DC Fan, a pouch 320 is sown on the fabric of the innermost layer of the face mask to hold the DC brushless motor and fan 330. The DC brushless motor helps in cooling the interior of the face mask as well as the user, since activating it allows more oxygen to be forced in the face mask whenever worn, preventing over exposure to CO2, due to inhalation & exhalation in an enclosed environment. It operates by a DC power source 360. For protecting the power source 360 an ampere fuse 350 is installed which prevents damage to the power source. The positive and negative wires 340 are camouflaged within the Velcro loop and hook fabric layer 251, 252 which runs peripherally around the mask as well is aligned longitudinally in the centre of the mask. The wires 340 are connected to the power source 360 through DC plugs 370 for operating the DC fan 330. A dimmer switch or speed control switch 365 is provided for adjusting the speed of the fan for increasing the air in the hot days or to decrease the air during winter months.

In some embodiments, Velcro loop and hook fabrics 251, 252 are sown on both sides of the small pouch 70 which is held at the upper part of the oval Face Mask which coincides with the bridge of the nose, for holding a small spring 80. This enables the small pouch 70 to be used when the Face Mask is turned around.

Referring to FIGS. 4, 251 and 252 are the Velcro loop and hook fabrics to be attached to the 252 and 251 respectively. Also depicted is a round hole copper rivets 255 and magnets 270 for trapping the positive and negative particulate matters in its magnetic field. The round hole copper rivets 255 allows the air to pass through the magnetic field generated by the magnet 270 for trapping the positive and negative particulate matters which helps in filtering the air before inhalation of the air by the user. Copper rivet holes 255 can be used to supply more air into the face mask when exercising or working. A fabric layer 256 is stitched on each layer of the hemp face mask which incorporates the copper rivet 255 and which acts as a one way valve for the copper rivet 255. The magnet 270 is placed in a pouch 260 which is sown on to the fabric layer 256. 280 and 290 are the Velcro loop and hook fabric respectively for covering the inlet copper rivet holes in a harsh environment by forcing air to filtrate through pouch 60 or to keep the Face Mask warmer in harsh weather conditions.

The magnets 270, and the created magnetic field around the copper rivet 255, the fabric layer 256, and the pouch 260 and their surroundings for trapping the positive and negative particulate matters in its magnetic field and also onto different layers of fabrics viz. the outer most layer 190, the intermediate layer 200, and the innermost layer 210 of the hemp mask.

The pouch 260 may be used to trap any type of air particles and moist particles in the air that will be trapped by using a magnetic field generated by the magnets 270. Once the magnets 270 are removed from the pouches inside the face mask, the face mask can be cleaned to remove any type of particles from the air that were collected onto the hemp fabric or other types of woven fibers or non woven materials that are washable, reusable and antibacterial.

The fabric layer 256 is used as a one way valve, for the copper rivet hole 255. The fabric layer 256 used for the one way valves may also be used for trapping any type of air particles and moist air particles in the air which may be trapped by using a magnetic field using magnets 270. Once the DC brushless motor and fans 330 and magnets 270 are removed from the pouches 260, the face mask can be cleaned to remove any type of particles from the air that were collected onto the hemp fabric or other types of fabric or material that are woven or non woven materials that are washable, reusable and antibacterial.

For keeping the pocket 60 open for breathing under normal conditions a cover 300 with sown on Velcro 251, 252 will attach to Velcro fabric 290.

The hook and the loop Velcro fabric which are depicted as 310 and 315 respectively, are used for attaching each other, thereby creating a direct air flow. This allows at least 20% of the Face Mask to channel out the carbon dioxide when closed, if the patient is healthy. If a patient is sick, the velcros 310 and 315 can open up by applying another velcro strip on 310 or 315, to allow the contaminated air particles from a sick patient to pass through layers of fabric that have a magnetic field surrounding them, to capture the positive and negative particles, thus enabling the exhaled air to pass out in the environment, devoid of its contaminations.

In some embodiments the DC fan along with the components and the copper rivets along with the magnets can be added to both the left and right side of the hemp face mask.

Referring to FIG. 6, which is an ear muffin 400 which can be attached to the face mask for providing warmth to the ears or for protecting the ear drums from loud noises.

The ear muffins are attached to the face mask by means of brass buttons, where 390 is the male brass buttons that will be used to fasten ear muffins 400 to the face mask. The female brass buttons 380 on the face mask are used to fasten with the male brass buttons 390 on the ear muffins 400.

The ear muffin is also made of multiple layers namely an outer layer 410 which is water proof and fire proof. Most preferably the fabric used is treated hemp fabric. A second layer 415 is a protective layer that helps to reduce sound noise to the ear drums. The innermost layer 420 which may comprise of fabric or animal fur like wool to provide warmth to the ears.

In some embodiments of the face masks for animals the same innovations as in case for humans the face masks for animals can also be incorporated with interchangeable multiple layers with Velcro loop and hook fabric 251, 252. The DC motor fan 330 is incorporated in a pouch 320 is sown on the fabric of the innermost layer of the face mask. The DC brushless motor helps in cooling the interior of the face mask as well as the user, since activating it allows more oxygen to be forced in the face mask whenever worn, preventing over exposure to CO2, due to inhalation & exhalation in an enclosed environment. It operates by a DC power source 360. For protecting the power source 360 an ampere fuse 350 is installed which prevents damage to the power source. The positive and negative wires 340 are camouflaged within the Velcro loop and hook fabric layer 251, 252 which runs peripherally around the mask as well is aligned longitudinally in the centre of the mask. The wires 340 are connected to the power source 360 through DC plugs 370 for operating the DC fan 330. A dimmer switch or speed control switch 365 is provided for adjusting the speed of the fan for increasing the air in the hot days or to decrease the air during winter months.

Since an average adult, as is known in the art, at rest inhales and exhales 7-8 litres (about one-fourth of a cubic foot) of air per minute. The DC fan of the present invention puts out 0.95 CFM, and the most for a small DC fan can put out 3.95 CFM that could fit in the face mask thereby allowing more oxygen to be inhaled during each inhalation. As is described in above embodiments, adding a second fan to the multilayered face mask, increases CFM air flow by 2 fold. In the present invention the DC fan can be of varying sizes depending upon the environment and the requirement.

While the invention has been described with respect to certain specific embodiments, it will be appreciated that many modifications and changes may be made by those skilled in the art without departing from the spirit of the invention. It is intended, therefore, by the appended claims to cover all such modifications and changes as fall within the true spirit and scope of the invention.

1. A multilayered hemp face mask with the flexibility of layer-interchangeability for filtering air particulate matter which has velcro loop and hook fabrics integrated with each of the fabric layers of the mask, the mask comprising;
at least one DC fan;
at least one copper rivet and a magnet;
at least two, one way fabric valves; and
a pair of ear muffins.

2. The multilayered mask of claim 1, wherein the said DC fan unit comprises of brushless motor and fan, a power source, an ampere fuse and a dimmer switch.

3. The multilayered mask of claim 1, wherein the said magnet is incorporated in a pouch sown on to the fabric layer functioning as a one way valve for trapping any type of air particulate matter and moist particles in the air which may be trapped by the magnetic field generated by the magnets.

4. The multilayered mask of claim 1, wherein the said copper rivet is covered by a fabric layer which also functions as a one way valve for trapping any type of air particulate matter and moist particles in the air which may be trapped by the magnetic field generated by the magnets.

5. The multilayered mask of claim 1, wherein the mask is washable and reusable.

6. The multilayered mask of claim 1, wherein the said DC fan is a 0.95 CFM.

7. The multilayer face mask of claim 1, wherein the said ear muffins are attached to the face mask by means of brass buttons.

8. The multilayer face mask of claim 1, wherein the said ear muffin comprises of:
an outer water proof and fire proof layer;
a second protective layer for reducing sound noise; and
an innermost layer of fabric or animal fur like wool for providing warmth to the ears.

9. The multilayer face mask of claim 1, wherein the face mask is designed to be worn by animals comprising of a brushless motor and fan, a power source, an ampere fuse and a dimmer switch.
